(19) United States
(12) Patent Application Publication
(10) Pub. No.: US 2008/0293774 A1
(43) Pub. Date: Nov. 27, 2008

(54) Treatment of the Symptoms of Bladder Irritation

(75) Inventors: Hugues Bienayme, St Symphorien D'Ozon (FR); Jacques Ferte, Issy-Les-Moulineuax (FR)

Correspondence Address: OLIFF & BERRIDGE, PLC, P.O. BOX 320850, ALEXANDRIA, VA, 22320-4850, US

(73) Assignee: UROGENE, PARIS (FR)

(21) Appl. No.: 12/083,246

(22) PCT Filed: Aug. 28, 2006
(86) PCT No.: PCT/IB2006/003691

§ 371 Date: May 8, 2008

Related U.S. Application Data
(60) Provisional application No. 60/727,830, filed on Oct. 19, 2005.

(30) Foreign Application Priority Data
Oct. 19, 2005 (FR) 05 10650

(51) Int. Cl. A61K 31/444; A61P 13/10

(52) U.S. Cl. 514/339

(57) ABSTRACT

Use of a compound corresponding to formula (I), for obtaining a medicinal product for use in the treatment of the symptoms of bladder irritation.

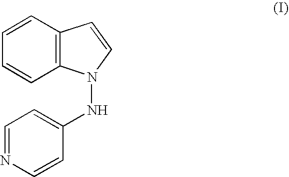

The present invention relates to the urological applications of compounds of N-(4-pyridinyl)-1H-indol-1-amine type, and more particularly to the use thereof in the treatment of the symptoms of bladder irritation associated with indications such as bladder instability (overactive bladder or OAB) or interstitial cystitis. These symptoms are frequency of micturition, urgency and nocturia.

The function of the lower urinary tract is to store and, when appropriate, release urine. Briefly, the bladder is a smooth muscle reservoir (the detrusor) that passively distends with filling. Closure of the bladder during the filling phase is secured by contraction of the urethral smooth muscle and of the external striated sphincter (rhabdosphincter). The lower urinary tract functions through a system of highly coordinated processes that involve the control of smooth and skeletal muscles of the bladder and urethra, by both central and peripheral nervous systems [Burgard et al, “New pharmacological treatments for urinary incontinence and overactive bladder”, Curr. Opin. Investig. Drugs. 6, 81-89 (2005)].

Under normal conditions, sensory information regarding bladder filling is primarily transmitted to the central nervous system (CNS) via Aδ sensory afferent fibers. When bladder volume has reached a critical threshold and micturition is behaviorally and environmentally appropriate, a spinobulbospinal reflex is activated which results in the release of acetylcholine at the bladder neuromuscular junction, producing a bladder contraction. Simultaneously, the urethra opens, following relaxation of both the smooth muscle and the rhabdosphincter and allows expulsion of the stored urine.

Impairment in the ability to store urine results in conditions which can be separated into two main disorders with completely distinct underlying dysfunctions: stress urinary incontinence and urge-related disorders.

Stress incontinence is a loss of urine in response to a cough, laugh, sneeze or any other physical activity that increases intra-abdominal pressure. The underlying pathology of stress urinary incontinence often involves the rhabdosphincter.

Overactive bladder (OAB) and the related condition of urge urinary incontinence represent the other major disorder of the storage function. They are characterized by a frequent need to void (frequency), with an intense urge to do so (urgency) and a need to void during the night (nocturia). Contrary to stress urinary incontinence, OAB and urge incontinence are not associated with urethral sphincter control but rather involves disturbances in bladder functions and the regulation thereof. In particular, an excessive stimulation of the afferent pathways is increasingly recognized as being an important element in the physiopathology of OAB [Kumar et al., “Recent advances in basic science for overactive bladder”, Curr. Opin. Urol. 15, 222-226 (2005)]. The conditions which can contribute to the appearance of these symptoms are, for example, ageing, urinary infections or an obstructive pathology such as benign prostatic hyperplasia.

In addition, these symptoms are associated with conditions involving irritation of the bladder urothelium, such as interstitial cystitis. Interstitial cystitis is a chronic, severely debilitating disease of the urinary bladder which, in addition to the OAB symptoms, is characterized by chronic pelvic pain with burning sensations and negative urine cultures.

Currently, the pharmacological treatments for stress urinary incontinence and urge-related disorders are completely distinct.

The pharmacological treatment of urge incontinence and OAB is based mainly on the use of medicinal products that act on the contractility of the bladder muscle, at the front line of which are the anti-muscarinics, such as oxybutynin, tolterodine, solifenacin or darifenacin. These medicinal products show a certain clinical efficacy on decreasing the number of urinary incontinence episodes. However, this class of medicinal products has no action on the afferent pathway, i.e. on the transmission of the nerve message to the central nervous system, and do not demonstrate, clinically, any beneficial effect on the urge phenomena. Moreover, anti-muscarinics have considerable side effects, related to their pharmacology, such as dryness of the mouth and blurred vision, which are the cause of the treatment being abandoned by patients in more than 50% of cases [see Herbison et al., “Effectiveness of anticholinergic drugs compared with placebo in the treatment of overactive bladder: systematic review”, British Medical Journal, 326, 841-844 (2003)].

These bladder conditions which affect all age categories have a harmful impact on the daily life of the individuals who suffer therefrom, and medicinal products which are more effective on the urge phenomena, of which it is possible to limit the number of doses and which exhibit little or no side effects, are still being sought.

Document WO 02/064126 discloses a compound of type N-(4-pyridinyl)-1H-indol-1-amine, N-(3-fluoro-4-pyridinyl)-N-propyl-3-methyl-1H-indol-1-amine or HP184, corresponding to the formula below

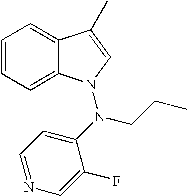

which, during in vivo trials carried out on rats suffering from bladder irritation, showed an effect by slightly decreasing the frequency of bladder contractions related to this condition.

The authors of the present invention have discovered that a set of N-(4-pyridinyl)-1H-indol-1-amine compounds, different from that which is the subject of WO 02/064126, have surprising properties with respect to the bladder sphere. The authors have demonstrated that these compounds act selectively on the afferent pathway resulting in the positive response of the bladder, and are found to be very effective in decreasing the manifestations associated with the abovementioned conditions.

Thus, the invention relates to a compound corresponding to formula (I) below:

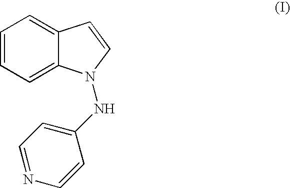

or N-(4-pyridinyl)-1H-indol-1-amine,

and also the pharmaceutically acceptable salts thereof, and more particularly the use thereof in obtaining a medicinal product for use in the treatment of the symptoms associated with bladder irritation.

The invention also relates, in the same use, to any compound corresponding to formula (II) below:

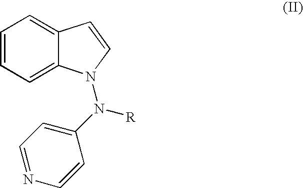

in which R is a group that is substituted with hydrogen by biotransformation, and also the pharmaceutically acceptable salts thereof.

According to the invention, the term “biotransformation” is intended to mean any biochemical conversion of said compound (II) in the human body that produces a metabolite, and more specifically compound (I). These conversions are essentially enzymatic, they may also be chemical, such as a hydrolysis in an acidic medium or an oxidation. In the case of the above compounds, given their structure, the substitution of the group R with hydrogen will generally occur during the phase I metabolism reactions. Biotransformation to compound (I) can be determined using in vitro tests with enzymatic preparations or in vivo pharmacokinetic experiments.

These compounds can therefore be considered as prodrugs of compound (I). Examples of suitable substituents can be found in Bundgaard, “Design of prodrugs”, Ed. Elsevier, 1985.

Thus, the group R will advantageously be chosen from alkyl, alkylene, alkylidene, cycloalkyl, cycloalkylene, cycloalkylidene and —CONH₂ groups, and —COR′ and —COOR′ groups, where R′ is chosen from alkyl, alkylene, alkylidene, cycloalkyl, cycloalkylene and cycloalkylidene groups, it being possible for said groups R and/or R′ to be substituted and/or interrupted with —O—, —COO—, —OCO—, —NHCO— or —CONH— functions.

According to the invention, the term “alkyl group” is intended to mean a linear or branched, monovalent, saturated hydrocarbon-based chain containing from 1 to 6 carbon atoms, representative elements of which are, for example, as follows: methyl, ethyl, n-propyl, isopropyl, n-butyl, sec-butyl, isobutyl, tert-butyl, pentyl and hexyl groups. The term “cycloalkyl group” is intended to mean a monovalent, saturated, cyclic hydrocarbon-based chain containing from 3 to 7 carbon atoms, and one (monocycloalkyl group) or more (polycycloalkyl group) rings. Representative elements of the monocycloalkyl groups are, for example, as follows: cyclopropyl, cyclobutyl, cyclopentyl, cyclohexyl and cycloheptyl groups. A representative element of the polycycloalkyl groups is, for example, norbornyl.

The terms “alkyl” and “cycloalkyl” as defined above keep the same definition when they integrate the name of a group, for example in —CO-alkyl groups.

The term “alkylene group” is intended to mean a linear or branched monovalent hydrocarbon-based chain which is unsaturated and comprises at least one double bond, and which contains from 2 to 6 carbon atoms, representative elements of which are, for example, vinyl, 1-propenyl, 2-propenyl, isopropenyl, butenyl, pentenyl and hexenyl groups. The term “alkylidene group” is intended to mean a linear or branched monovalent hydrocarbon-based chain which is unsaturated and comprises at least one triple bond, and which contains from 2 to 6 carbon atoms, representative elements of which are, for example, ethynyl and propargyl groups.

The pharmaceutically acceptable salts of these compounds are also part of the invention. It may in fact be preferable to prepare, purify and/or store a salt corresponding to the active compound, for example a pharmaceutically acceptable salt. Examples of pharmaceutically acceptable salts are given in the publication Berge et al., “Pharmaceutically acceptable salts”, J. Pharm. Sci., 66, 1-19 (1977). By way of examples, mention may be made of salicylic, hydrochloric or fumaric-salts.

A particularly effective compound is the compound of formula (II) in which R is the n-propyl group. This compound, besipirdine, has been shown to be metabolized in human to compound (I) above [R S Hsu, E M DiLeo, S M Chesson, J T Klein, R C Effland, “Determination of HP749, a potential therapeutic agent for Alzheimer's disease, in plasma by high-performance liquid chromatography”, J. Chromatogr. 572, 352-359 (1991)]. This compound is all the more beneficial since it is known, according to the publication “Anti-obsessional and anti-depressant profile of besipirdine” by C. P. Smith et al. CNS Drug Review, 3, 1-23 (1997), that it is advantageous in the treatment of depression, which often accompanies bladder conditions.

The compounds (I) and (II) of the invention can be obtained by means of a process such as that described in U.S. Pat. No. 4,970,218.

A medicinal product or pharmaceutical composition of the invention is advantageously in a pharmaceutical form intended for oral administration or for parenteral administration.

The advantages of the compounds of the invention emerge from the examples disclosed hereinafter in support of the following figures:

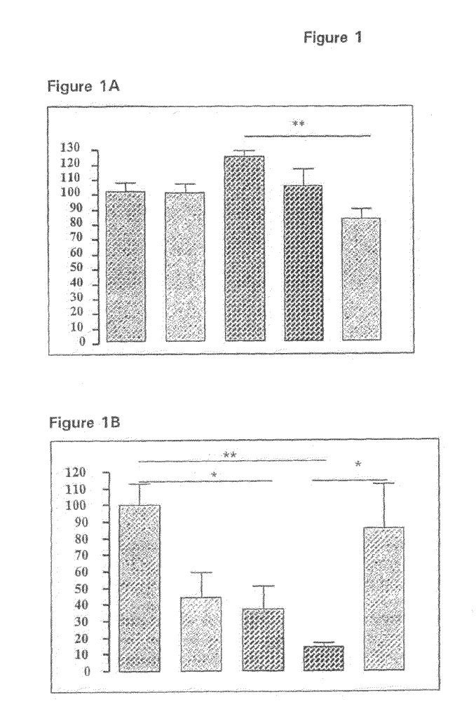

FIG. 1 illustrates the effect of the compound HP748 compared with that of oxybutynin, on maximum intravesical pressure (expressed as % relative to the carrier) according to FIG. 1A and on the frequency of rhythmic isovolumetric contractions (expressed as number of contractions per hour, as % relative to the carrier) according to FIG. 1B; on each of FIGS. 1A and 1B, from left to right, the histograms correspond, respectively, to the carrier, to HP748 at 0.3 mg/kg, to HP748 at 0.9 mg/kg, to HP748 at 3.5 mg/kg and to oxybutynin at 1 mg/kg;

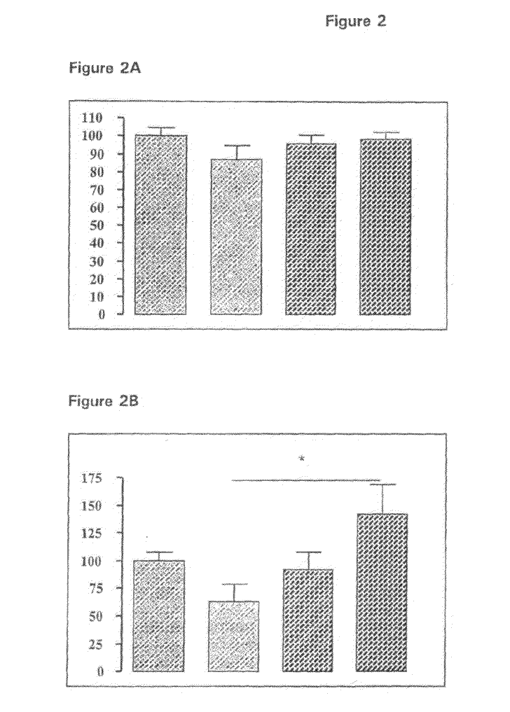

FIG. 2 illustrates the effect of the compound HP749 on maximum intravesical pressure (expressed as % relative to the carrier) according to FIG. 2A and on the frequency of rhythmic isovolumetric contractions (expressed as number of contractions per hour, as % relative to the carrier) according to FIG. 2B; on each of FIGS. 2A and 2B, from left to right, the histograms correspond, respectively, to the carrier, to HP749 at 1.6 mg/kg, to HP749 at 3.5 mg/kg and to HP749 at 10.5 mg/kg;

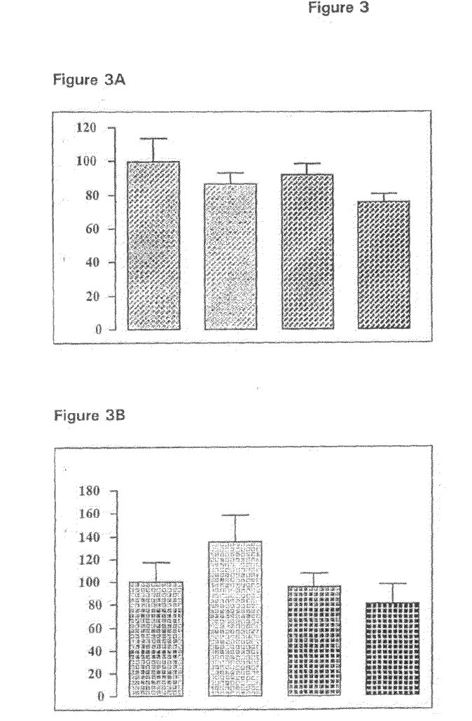

FIG. 3 illustrates the effect of the compound HP183 on maximum intravesical pressure (expressed as % relative to the carrier) according to FIG. 3A and on the frequency of rhythmic isovolumetric contractions (expressed as number of contractions per hour, as % relative to the carrier) according to FIG. 3B; on each of FIGS. 3A and 3B, from left to right, the histograms correspond, respectively, to the carrier, to HP183 at 0.3 mg/kg, to HP183 at 1 mg/kg and to HP183 at 3 mg/kg;

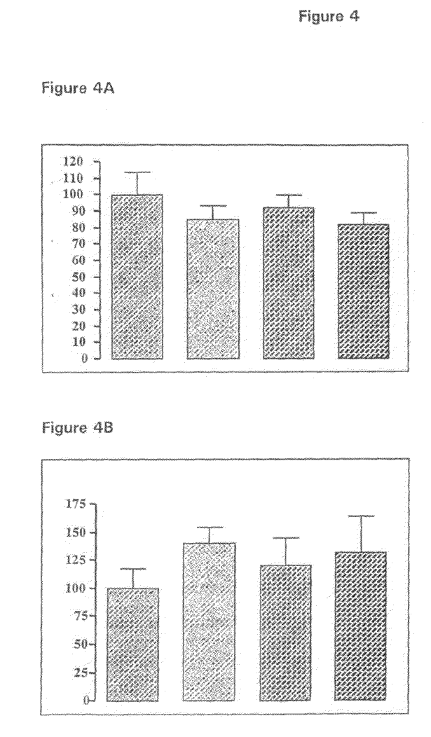

FIG. 4 illustrates the effect of the compound HP184 on maximum intravesical pressure (expressed as % relative to the carrier) according to FIG. 4A and on the frequency of rhythmic isovolumetric contractions (expressed as number of contractions per hour, as % relative to the carrier) according to FIG. 4B; on each of FIGS. 4A and 4B, from left to right, the histograms correspond, respectively, to the carrier, to HP184 at 1 mg/kg, to HP184 at 3 mg/kg and to HP184 at 10 mg/kg.

The examples hereinafter illustrate the effect of the compounds according to the invention and the advantage of the latter compared with known compounds that find applications in the urological field. Thus, the compounds tested are those described in Table 1 below, where the compound HP184 is that described in document WO 02/064126, and the compound HP183 is a metabolite of HP184.

TABLE 1

| Compounds tested | Formulae |
|---|---|
| HP748 (compound I of theinvention, main metabolite ofHP749)or N-(4-pyridinyl)-1H-indol-1-amine | 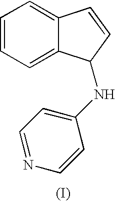 |
| HP749 (compound II of theinvention where R representsCH₂—CH₂—CH₃)or N-propyl-N-(4-pyridinyl)-1H-indol-1-amine | 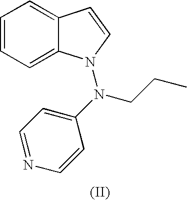 |
| HP183 (metabolite of HP184)or N-(3-fluoro-4-pyridinyl)-3-methyl-1H-indol-1-amine | 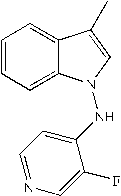 |
| HP184or N-(3-fluoro-4-pyridinyl)-N-propyl-3-methyl-1H-indol-1-amine | 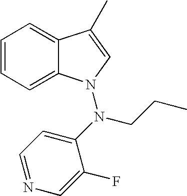 |
| (+/−) Oxybutynin (reference anti-muscarinic compound) | 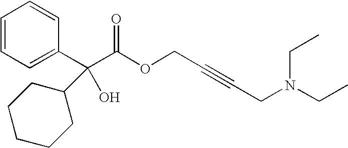 |

The compounds of Table 1 have been evaluated in a conventional animal model of overactive bladder: the complete bladder-outlet obstruction model in the rat, referred to as “isovolumetric” model (K. Sugaya, Y. Ogawa, O, Nishizawa, W C De Groat, Decrease in intravesical saline volume during isovolumetric cystometry in the rat, Neurourol. Urodyn, 1997, 16:125-132). In this model, rhythmic reflex contractions of the bladder are induced in female Wistar rats in which the urethra has been ligatured.

The rats are anaesthetized and implanted with a jugular catheter for the intravenous administration of the compounds. A transurethral catheter is introduced into the bladder, through the bladder dome, so as to allow the infusion of a saline solution and measurement of the intravesical pressure. The urethra is ligatured in order to close off the bladder.

The bladder is infused (50 μl/min) via the transurethral catheter with a saline solution until uninhibited rhythmic contractions appear. The infusion is then stopped and the intravesical pressure is measured for a period of 60 min preceding the injection of the study compounds. The compounds to be studied are then injected via the jugular catheter, and the intravesical pressure is recorded for a period of 60 min. The intravenous administration of the compounds during this test limits the metabolization of HP749 and of HP184 to HP748 and HP183, respectively. The animals are sacrificed at the end of the experiment by means of an overdose of pentobarbital. A scheme of the experiment is given below.

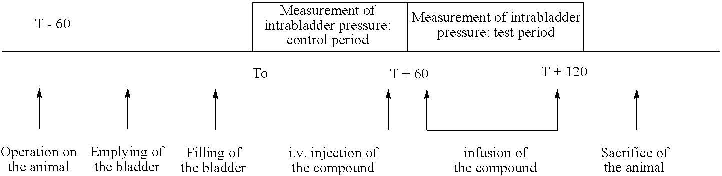

The data from the experiment are analysed with the ELPHY software (a free version of which is accessible at the following address: http://www.unic.cnrs-gif.fr/software/software.htm). The parameters analysed are as follows:
Maximum intravesical pressure (MP, expressed in mmHg)
Threshold pressure (ThP, expressed in mmHg) compared with the carrier (FIG. 2B).

HP183 (0.3, 1 and 3 mg/kg) and HP184 (1, 3 and 10 mg/kg) showed no effect, either on the maximum pressure or on the frequency of rhythmic isovolumetric contractions.

Conclusion of the Experiments

In the above experiments, HP184 and its metabolite, HP183, have no statistically significant effect, either on the maximum pressure or on the frequency of bladder contractions (FIGS. 3 and 4).

HP749 showed a slight effect on the frequency, decreasing the latter at the lowest dose and increasing it at the highest dose (10.5 mg/kg; i.v.). HP749 did not demonstrate any effect on the maximum pressure.

On the other hand, HP748 considerably and statistically significantly decreased the frequency of bladder contractions, from the first dose of 0.3 mg/kg. This effect is dose-dependent (FIG. 2B).

Despite the structural similarity of the four compounds tested, HP748 is the only compound of the N-(4-pyridinyl)-1H-indol-1-amine family to have demonstrated a significant activity in the isovolumetric model of overactive bladder. Its action on bladder contraction frequency demonstrates that it acts rather on the afferent pathway than on the efferent pathway. It therefore differs clearly, by virtue of its method of action, from the anti-muscarinic compounds such as oxybutynin.

Moreover, HP748 also demonstrated its unexpected superiority over other products of the same class, such as HP184 and HP183.

1. A method of treatment of symptoms of bladder irritation, the method comprising:
administering a medicinal product corresponding to formula (I): 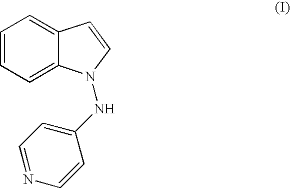
or a pharmaceutically acceptable salt of said compound, to a subject that has symptoms of bladder irritation.

2. A method of treatment of symptoms of bladder irritation, the method comprising:
administering a medicinal product corresponding to formula (II): 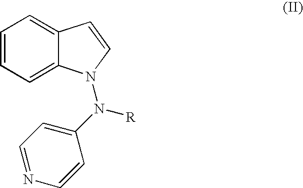
in which R represents a group that is substituted with hydrogen by biotransformation, or a pharmaceutically acceptable salt of said compound, to a subject that has symptoms of bladder irritation.

3. The method according to claim 2, wherein R is chosen from alkyl, alkylene, alkylidene, cycloalkyl, cycloalkylene, cycloalkylidene and —CONH₂ groups, and —COR′ and COOR′ groups, where R′ is chosen from alkyl, alkylene, alkylidene, cycloalkyl, cycloalkylene and cycloalkylidene groups, it being possible for said groups R and/or R′ to be substituted and/or interrupted with —O—, —COO—, —OCO—, —NHCO— or —CONH— functions.

4. The method according to claim 3, wherein R is the n-propyl group.

5. The method according to claim 1, wherein the symptoms of bladder irritation are bladder instability (overactive bladder, OAB) or interstitial cystitis.

6. The method according to claim 2, wherein the symptoms of bladder irritation are bladder instability (overactive bladder, OAB) or intestinal cystitis.

7. The method according to claim 3, wherein the symptoms of bladder irritation are bladder instability (overactive bladder, OAB) or intestinal cystitis.

8. The method according to claim 4, wherein the symptoms of bladder irritation are bladder instability (overactive bladder, OAB) or intestinal cystitis.
